(19) United States
(12) Patent Application Publication
(10) Pub. No.: US 2006/0165596 A1
(43) Pub. Date: Jul. 27, 2006

(54) Method and apparatus for noninvasively evaluating endothelial function

(76) Inventors: Nachiket Kharalkar, Austin, TX (US); Morteza Naghavi, Houston, TX (US)

Correspondence Address: DUANE, MORRIS, LLP, 3200 SOUTHWEST FREEWAY, SUITE 3150, HOUSTON, TX, 77027, US

(21) Appl. No.: 10/525,255

(22) PCT Filed: Aug. 22, 2003
(86) PCT No.: PCT/US03/26238

§ 371 Date: Feb. 23, 2005

Related U.S. Application Data
(60) Provisional application No. 60/405,352, filed on Aug. 23, 2002.

(51) Int. Cl. A61K 49/00

(52) U.S. Cl. 424/9.2; 424/9.5

(57) ABSTRACT

The present invention relates to assessing and measuring endothelial functions. A vasodilating stimulant is provided to a patient to stimulate hemodynamic activity in a selected region of the patient's body. In an embodiment, endothelial function may then be assessed by monitoring a change in a hemodynamic parameter at the selected region and assessing the patient's endothelial function based upon that monitoring. In a further embodiment, endothelial function may then be measured by monitoring a change in blood oxygen content at the selected region and assessing the patient's endothelial function based upon that monitoring. In yet a further embodiment, endothelial function may then be measured by monitoring a change in blood flow rate at the selected region and assessing the patient's endothelial function based upon said monitoring. It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

### PRIORITY

This application claims the benefit of U.S. Provisional Patent Application Ser. No. 60/405,352 filed on Aug. 23, 2002.

### FIELD OF INVENTION

The present invention relates generally to the field of assessing a patient's endothelial function by monitoring changes in hemodynamic parameters responsive to the introduction of a vasodilating stimulant. The monitored hemodynamic parameters may include blood temperature, blood flow, and/or blood oxygen content.

### BACKGROUND OF THE INVENTION

Cardiovascular disease and its sequel account for a significant percentage of the morbidity or mortality in industrialized countries. It is known that cardiovascular disease may be caused and/or enhanced by an impairment of tissue perfusion.

The endothelium has many important functions in maintaining the patency and integrity of the arterial system. The endothelium can reduce and inactivate toxic super-oxides which may be present in diabetics and in smokers. The endothelium is the source of nitric oxide, a local hormone that relaxes the adjacent smooth muscle cells in the media, and is a powerful vasodilator.

The endothelium regulates vascular homeostasis by elaborating a variety paracrine that act locally in the blood vessel wall and lumen. Under normal conditions, these aspects of the endothelium, hereinafter referred to as “endothelial factors,” maintain normal vascular tone, blood fluidity, and limit vascular inflammation and smooth muscle cell proliferation.

When coronary risk factors are present, the endothelium may adopt a phenotype that facilitates inflammation, thrombosis, vasoconstriction, and atherosclerotic lesion formation. In human patients, the maladaptive endothelial phenotype manifests itself prior to the development of frank atherosclerosis and is associated with traditional risk factors such as hypercholesterolemia, hypertension, and diabetes mellitus. The maladaptive endothelial phenotype is further identified with emerging risk factors such hyperhomocystinemia, obesity, and systemic inflammation.

Prior art means for estimating endothelial dysfunction include the use of cold pressure tests by invasive quantitative coronary angiography and the injection of radioactive material and subsequent tracking of radiotracers in the blood. These invasive methods are costly, inconvenient, and must be administered by highly trained medical practitioners.

Noninvasive prior art methods for measuring endothelial dysfunction include, the measurement of the percent change and the diameter of the left main trunk induced by cold pressure test with two dimensional echo cardiography, the Dundee step test, laser doppler perfusion imaging and iontophoresis, and high resolution b-mode ultrasound.

### SUMMARY OF THE INVENTION

In an embodiment, endothelial function may be assessed by providing a vasodilating stimulant to a patient to stimulate hemodynamic activity in a selected region of the patient's body; monitoring a change in a hemodynamic parameter at the selected region; and assessing the patient's endothelial function based upon said monitoring.

In a further embodiment, endothelial function may be measured by providing a vasodilating stimulant to a patient to stimulate hemodynamic activity in a selected region of the patient's body; monitoring a change in blood oxygen content at the selected region; and assessing the patient's endothelial function based upon said monitoring.

In yet a further embodiment, endothelial function may be measured by providing a vasodilating stimulant to a patient to stimulate hemodynamic activity in a selected region of the patient's body; monitoring a change in blood flow rate at the selected region; and assessing the patient's endothelial function based upon said monitoring.

It is emphasized that this summary is not to be interpreted as limiting the scope of these inventions which are limited only by the claims herein.

### BRIEF DESCRIPTION OF THE DRAWINGS

FIG. 1 is a flowchart of a first preferred embodiment of a method of endothelial function assessment;

FIG. 2 is a flowchart of a second preferred embodiment of a method of endothelial function measurement; and

FIG. 3 is a flowchart of a second preferred embodiment of a method of endothelial function measurement.

### DETAILED DESCRIPTION OF PREFERRED EMBODIMENTS OF THE INVENTION

As used herein, that which is described as software may be equivalently implemented as hardware.

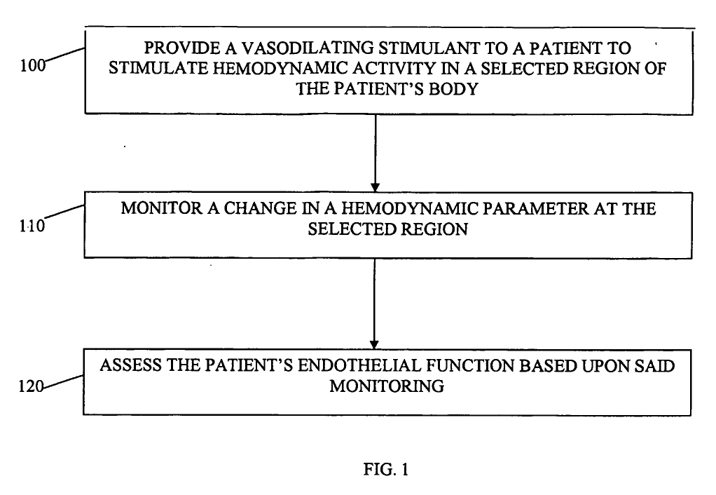

Referring now to FIG. 1, a preferred method for assessing endothelial function comprises providing a vasodilating stimulant to a patient to stimulate hemodynamic activity in a selected region of the patient's body, illustrated at block 100 in FIG. 1; monitoring a change in a hemodynamic parameter at the selected region, illustrated at block 110 in FIG. 1; and assessing the patient's endothelial function based upon said monitoring, illustrated at block 120 in FIG. 1. In a preferred embodiment, the monitored hemodynamic parameter may be a parameter such as blood temperature, blood oxygen content, blood flow rate, or the like, or a combination thereof.

Providing a vasodilating stimulant may further comprise compressing the patient's brachial artery for a predetermined period of time and ceasing the compression after that predetermined period of time. Providing a vasodilating stimulant may also comprise occluding blood flow in the patient's arm.

Additionally, the change in temperature at one of the patient's fingertips may be monitored as may the change in temperature in the patient's arm. Monitoring the change in temperature may be accomplished by placing at least two temperature sensors, for example piezoelectric sensors, proximate, e.g. on, the patient's forearm. The temperature sensors may be separated by a known distance.

Providing a vasodilating stimulant may comprise occluding blood flow in the patient's leg.

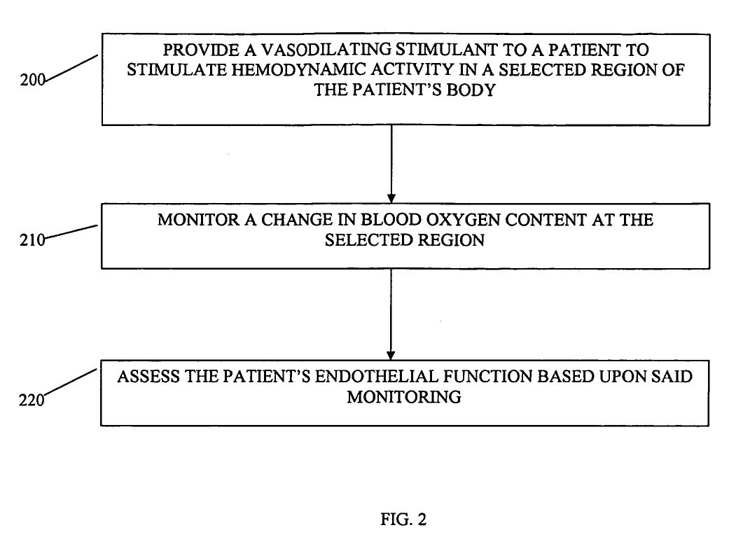

Referring now to FIG. 2, in a preferred method for measuring endothelial function comprises providing a vasodilating stimulant to a patient to stimulate hemodynamic activity in a selected region of the patient's body, illustrated at block 200 in FIG. 2.; monitoring a change in blood oxygen content at the selected region, illustrated at block 210 in FIG. 2.; and assessing the patient's endothelial function based upon said monitoring, illustrated at block 210 in FIG. 2.

Monitoring may be accomplished by taking measurements with a pulse oximeter. The pulse oximeter may be placed proximate, e.g. on, the tip of one of the patient's fingers.

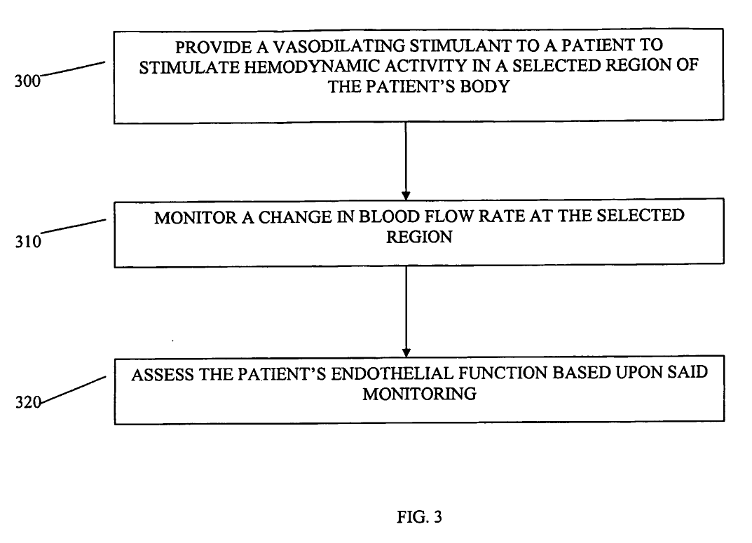

Referring now to FIG. 3, a second preferred method for measuring endothelial function comprises providing a vasodilating stimulant to a patient to stimulate hemodynamic activity in a selected region of the patient's body, illustrated at block 300 in FIG. 3; monitoring a change in blood flow rate at the selected region, illustrated at block 310 in FIG. 3; and assessing the patient's endothelial function based upon said monitoring, illustrated at block 320 in FIG. 3.

Monitoring may be accomplished by taking measurements with a photoplethysmograph placed proximate, e.g. on, one of the patient's fingers. Monitoring may also be accomplished by taking an ultrasound Doppler measurement. Monitoring may occurs from a time prior to the beginning of the compression until a time after ceasing, e.g. when blood flow has stabilized.

Providing a vasodilating stimulant may comprise compressing one of the patient's arteries located in an outer extremity of the patient's body for a predetermined period of time and ceasing the compression after said predetermined period of time. The outer extremity may be a leg, an arm, a wrist, and/or a finger.

The second preferred method for measuring endothelial function may further comprise plotting measured blood flow as a function of time and/or plotting the change in blood flow as a function of time.

It will be understood that various changes in the details, materials, and arrangements of the parts which have been described and illustrated above in order to explain the nature of this invention may be made by those skilled in the art without departing from the principle and scope of the invention as recited in the appended claims.

### STATEMENT OF INDUSTRIAL USE

The present invention may be used to assess a patient's endothelial function by monitoring changes in hemodynamic parameters responsive to the introduction of a vasodilating stimulant. The monitored hemodynamic parameters may include blood temperature, blood flow, and/or blood oxygen content.

What is claimed is:

1. A method for assessing endothelial function, comprising:
a. providing a vasodilating stimulant to a patient to stimulate hemodynamic activity in a selected region of the patient's body;
b. monitoring a change in a hemodynamic parameter at the selected region; and
c. assessing the patient's endothelial function based upon said monitoring.

2. The method of claim 1, wherein providing a vasodilating stimulant comprises:
a. compressing the patient's brachial artery for a predetermined period of time; and
b. ceasing said compression after said predetermined period of time.

3. The method of claim 2, wherein said monitoring further comprises monitoring a change in temperature at one of the patient's fingertips.

4. The method of claim 1, wherein providing a vasodilating stimulant comprises occluding blood flow in the patient's arm.

5. The method of claim 4, wherein said monitoring comprises monitoring a change in temperature in the patient's arm.

6. The method of claim 5, wherein monitoring the change in temperature in the patient's arm is accomplished by placing at least two temperature sensors proximate the patient's forearm.

7. The method of claim 6, wherein the temperature sensors are piezoelectric sensors.

8. The method of claim 1, wherein the hemodynamic parameter is at least one of (i) blood temperature, (ii) blood oxygen content, or (iii) blood flow rate.

9. The method of claim 1, wherein providing a vasodilating stimulant comprises occluding blood flow in the patient's leg.

10. A method for measuring endothelial function, comprising:
a. providing a vasodilating stimulant to a patient to stimulate hemodynamic activity in a selected region of the patient's body;
b. monitoring a change in blood oxygen content at the selected region; and
c. assessing the patient's endothelial function based upon said monitoring.

11. The method of claim 10, wherein said monitoring is accomplished by taking measurements with a pulse oximeter.

12. The method of claim 11, wherein said pulse oximeter is placed proximate the tip of one of the patient's fingers.

13. A method for measuring endothelial function, comprising:
a. providing a vasodilating stimulant to a patient to stimulate hemodynamic activity in a selected region of the patient's body;
b. monitoring a change in blood flow rate at the selected region; and
c. assessing the patient's endothelial function based upon said monitoring.

14. The method of claim 13, wherein said monitoring is accomplished by taking measurements with a photoplethysmograph placed proximate one of the patient's fingers.

15. The method of claim 13, wherein said monitoring is accomplished by taking an ultrasound Doppler measurement.

16. The method of claim 13, wherein providing a vasodilating stimulant comprises:
a. compressing one of the patient's arteries located in an outer extremity of the patient's body for a predetermined period of time; and
b. ceasing said compression after said predetermined period of time.

17. The method of claim 16, wherein the extremity is at least one of (i) a leg, (ii) an arm, (iii) a wrist, of (iv) a finger.

18. The method of claim 17, wherein said monitoring occurs from a time prior to the beginning of said compression until a time after said ceasing when said blood flow has stabilized.

19. The method of claim 18, further comprising plotting measured blood flow as a function of time.

20. The method of claim 19, further comprising plotting the change in blood flow as a function of time.
